(19) United States
(12) Patent Application Publication
(10) Pub. No.: US 2004/0196984 A1
(43) Pub. Date: Oct. 7, 2004

(54) Dynamic noise suppression voice communication device

(76) Inventors: Stephen G. Dame, Everett, WA (US); Allan Prince, Kirkland, WA (US); Paul Brickhouse, Redmond, WA (US)

Correspondence Address: GRAYBEAL, JACKSON, HALEY LLP, 155 - 108TH AVENUE NE, SUITE 350, BELLEVUE, WA, 98004-5901, US

(21) Appl. No.: 10/625,461

(22) Filed: Jul. 22, 2003

Related U.S. Application Data
(60) Provisional application No. 60/397,937, filed on Jul. 22, 2002.

(51) Int. Cl. A61F 11/06; H03B 29/00; H04M 1/00

(52) U.S. Cl. 381/71.1; 379/392.01

(57) ABSTRACT

A fast acting noise suppression device that uses energy level (volume level) of the input signal averaged over six to twenty milliseconds as a gain control for an output amplifier. The device is implemented with a digital signal processor (DSP). Rather than using an arithmetic formula to relate input volume to gain control for the amplifier, a look up table is used, allowing precise control of the function between input volume and amplifier gain. The range of lower input volumes which are suppressed by receiving relatively less amplification can be adjusted with a manual user control. The quality of the sound output as perceived by a listener is further improved by compression of high volume inputs so that they receive relatively less amplification than medium volume inputs.

This application claims priority from U.S. provisional patent application No. 60/397,937 filed Jul. 22, 2002, which is incorporated herein by reference.

### FIELD OF THE INVENTION

The present invention pertains generally to voice communications equipment, and more particularly to a device for suppressing ambient noise picked up by a microphone when the microphone is in a moderate to high noise environment.

### BACKGROUND

Many situations exist where humans use communications devices that provide a microphone input near their head and one or two sound transducers near their ears such as earpieces, headphones, or other speakers. Most often, the devices are being used inside noisy vehicles, confined areas next to machines, motors, etc. or other inside or outside environments where a broadband noise source exists across the audio frequency spectrum.

A few companies such as Bose, Sennheiser and Telex have successfully produced noise-canceling headphones which produce very good suppression of sound waves that penetrate typical earphone cups used in headsets. These earphone noise cancellation techniques use methods to sense noise in the proximity of the ear and inject anti-noise sound into the same ear proximity area to actively cancel the penetrating sound waves.

Other companies, such as Sennheiser and Gentex Corporation, have produced common mode noise canceling microphones that suppress lower frequency energy present at both front and back sides of a microphone when a voice input is present on only one side of the microphone. However, due to phase relationships of many of the higher frequency components of sound entering the front of the microphone compared to the back of the microphone, it is difficult or impossible to remove all of the objectionable frequencies and much of this noise is still present at the output of the microphone. This remaining noise is a source of fatigue in noisy environments such as flying commercial and private aircraft, operating heavy machinery, participating as a crew member of a military vehicle such as a tank, riding motorcycles outdoors, etc.

In the prior art, there are numerous examples of a voice activated switch device (VOX) that attempts to turn on a voice communications channel at a variable input threshold so that background noise between spoken words is suppressed. This is problematic for a few reasons. First, the first syllable of a spoken utterance is frequently muted while the VOX switch is detecting the voice energy threshold sufficiently for turning on the voice switch. Second, the time when the VOX should switch off is difficult to determine, so most devices simply wait for a fraction of a second to a couple of seconds to turn off the switch. This enables the ambient noise to remain in the output audio after the spoken words have ceased. Third, in the case of aircraft for example, when a threshold for the VOX switch has been set on the ground when the pilot is experiencing low engine RPM's and noise, it frequently needs to be adjusted with different aircraft power settings as the aircraft becomes airborne and goes thru various power settings and different noise sound pressure levels are applied to the microphone.

### SUMMARY OF THE INVENTION

The present invention relates to a device that dynamically applies the energy of the voice as a control signal to modulate the volume of an input microphone signal to achieve dynamic voice activated noise suppression. When the energy of the microphone signal is low, very little amplification energy is applied to boost the volume of the microphone signal. If the energy is medium to high, amplification energy is applied to the microphone output sufficient to raise the signal level to audible levels. The perceptual effect of this is that the ambient noise appears (to the listener) to be removed from the signal. This is due to the psychoacoustic effect that louder signals tend to mask softer signals (even if the softer signals are noise). Generally, even in a high noise environment, the energy of the noise signal is somewhat lower than the direct spoken input to a microphone, due to the proximity of the typical microphone to the speaker's mouth. When the person stops speaking, the volume of the amplified noise input immediately (within 6-20 milliseconds) tracks the voice energy downward and is thus perceived by the listener to be suppressed immediately after the speaker finished their spoken utterances.

The present invention directly extracts the energy (or absolute amplitude averaged over a short period of time—6-20 milliseconds) from the voice in a linear fashion and then applies a non-linear transfer function to the voice energy to further enhance the contrast between the low level undesirable signals and the higher level voice signals. Instead of switching suddenly on or off like the prior art VOX systems, the output volume level changes gradually (smoothly) and continuously (no sudden jumps) as the input volume level changes. As averaged over a very short period of time, low input volumes are suppressed, medium-low volumes are unchanged, mid to high level volumes are boosted, and the transitions are gradual and continuous. As a further refinement, the high level volumes may be unchanged such that only the mid level volumes are boosted. This further improves clarity of voice communications.

The present invention applies a parallel approach to achieve the signal processing objectives whereby the signal energy E is calculated in one path and the original input signal X is passed through on a second path but is modified by a signal volume control element 38 (such as a gain multiplier at the end of the path) as shown in block diagram form in FIG. 2b.

One embodiment of the present invention is a multi-channel interphone system for small aircraft that can support as many as 6 stereo headset/microphone sets as well as CD/DVD audio inputs, recorder outputs, cellular telephone inputs and outputs, and a direct connection to a two-way aircraft VHF radio system. In this embodiment, a Digital Signal Processing microprocessor (DSP) is used to provide the necessary switching, mixing and application of the software algorithm of the present invention to perform the dynamic voice activated noise suppression. In this system, each microphone input has independent voice activated noise suppression applied and the outputs are summed as appropriate to whatever sources are selected for the intercom.

In summary, in various aspects, the invention may be characterized as a device which implements the following methods:

1) A noise reduction method in which a suppressed output signal Y is calculated by extracting from a microphone input signal X a dynamic energy signal E which is averaged over a short period of time (smoothed), and applying this dynamic energy signal E to the original input microphone signal X as a volume control.

2) A method within method (1) which produces a smooth (gradual and continuous) energy function E which is used as a dynamic volume control signal.

3a) A method within method (2) which adjusts the sensitivity of the energy computation by adjusting the level of the input microphone signal X before it is applied to the signal energy computation method.

3b) A method within method (2) which computes a full-wave rectification (absolute value of the input signal on a sample by sample basis) of the input sensitivity adjusted audio microphone signal Xg to produce a coarse energy function Ec.

3c) A method within method (2) which smoothes (averages over a short period of time) the coarse full-wave rectified signal Ec to produce a linear output energy function Es of the input signal Xg.

3d) A method within method (2) which transforms the smoothed linear energy function Es into an optimized energy function E which is to be applied to the input signal X to suppress background noise. This transformation can be any input to output transfer function, but in a preferred embodiment it is a lookup table that enhances voice level signals and suppresses lower level background noise signals.

3(e) A method within (3d) where the transfer function performs an expansion of low volume (noise only) signals and a compression of mid to high volume signals so that high volume signals receive less amplification than medium volume signals.

4) A method within method (2) which provides a variable control to blend the amount of noise suppression energy signal E with a simple volume level to give the user a choice of how much of the original input signal X they wish to hear blended with the noise suppression control of the input signal X. In one embodiment, this method uses the maximum of either the energy signal E or the static input level control set by the user which is then directly multiplied times the input signal to obtain the output noise suppressed signal Y. Because the maximum of these two sources is used, when the user sets the input high, there is no input signal modification; when the user sets the input low, there is full input signal modification; and when the user sets the input medium, the low level signal expansion (suppression) is less effective compared to when the user sets the input low. However, the effect of setting to medium causes the lower level signals to still get multiplied by a smaller number than full scale volume while still allowing the medium to high voice signals to pass at their envelope tracked higher volumes. The highest volumes are still compressed and the mid level volumes are boosted which gives rise to better intelligibility.

In another aspect, the invention is an interphone communication system which incorporates a plurality of noise reduction methods above, one to each of a plurality of user microphone inputs, and provides these noise reduced voice signals to various output sources such as multiple intercom network headphones, VHF radio inputs and other two-way communications devices such as cellular telephones

### BRIEF DESCRIPTION OF THE DRAWINGS

FIG. 1 shows a system composed of a DSP, Flash memory that contains the DSP program, a bank of CODEC (coders and decoders) (stereo analog to digital converts, and digital to analog converters) circuits and necessary input and output amplifiers for general analog signal conditioning.

FIG. 2a is a signal flow path diagram for the present invention whereby an input digital audio microphone signal X is applied to a dynamic voice activated noise suppression filter algorithm to produce an output signal Y.

FIG. 2b shows how the input signal X is multiplied by an energy control signal E applied to a volume control element 38 to produce a dynamic voice activated noise suppression processed output Y.

FIG. 2c is an internal signal flow diagram showing how the signal energy function E is derived from input signal X.

FIG. 3 shows a typical amplification curve as a function of volume averaged over a short period of time.

### DETAILED DESCRIPTION

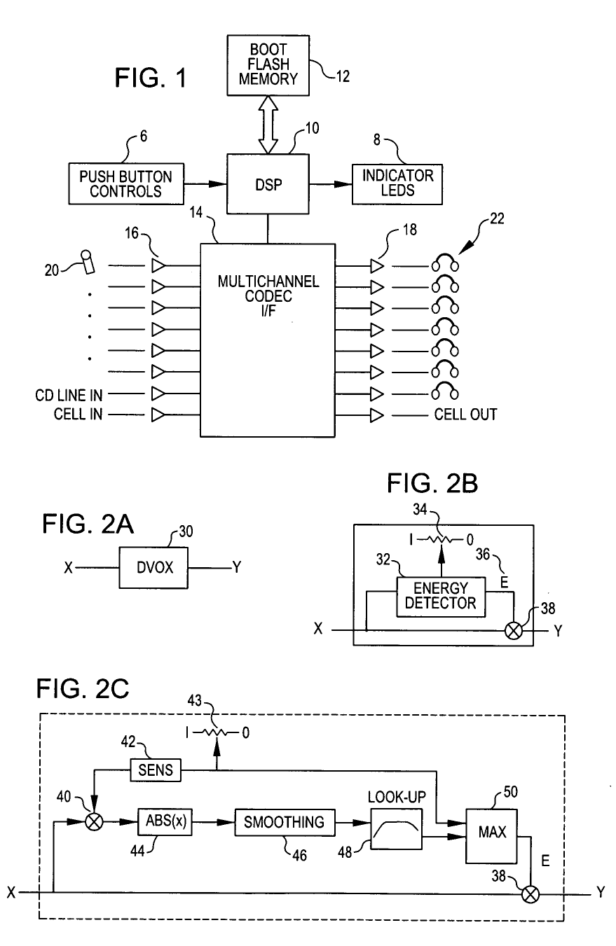

FIG. 1 shows a block diagram for a small general aviation intercom processing system according to the present invention. This system includes user controls 6, out put LED's 8, a Digital Signal Processing CPU (DSP) 10, Flash memory 12, a multichannel stereo CODEC 14, microphone/line preamps 16, headphone/line amplifiers 18, input jacks for microphones 20 and output jacks for headsets/speakers 22.

FIG. 2 shows different levels of detail of the noise suppression circuit block diagram. FIG. 2a shows the high level block diagram flow of the input signal X thru the dynamic voice activated noise suppression filter to form the output signal Y.

FIG. 2b shows, in a functional diagram, the parallel structure of the signal flow whereby the input signal X is passed through to a single output multiplier 38 which applies the detected energy function E (volume) of the input signal X to the output multiplier 38. A manual user controlled variable level function 34 is applied to the energy detection process to optimize the energy detection sensitivity and/or blend the amount of signal bypass that the user may desire.

FIG. 2c shows the internal functional details of an embodiment of the invention including the energy detector, sensitivity adjustment, and bypass operation functions. The input signal X is gain adjusted, function 40, via a sensitivity mapping function 42 and then passed through a full-wave rectification process 44 (i.e. absolute value of (x)) in order to obtain a coarse linear representation Ec of the (sensitivity gain adjusted) input speech plus noise signal volume. In a preferred embodiment, this coarse signal Ec is passed through two cascaded efficient low pass smoothing filters 46 (i.e. box car average) each of which averages the coarse energy signal Ec over 8 milliseconds. Because each averaging filter introduces a delay equal to one half of the duration that is averaged, the two filters produce a smoothed output Es which is approximately delayed from the input signal by about 8 milliseconds.

The design objective is to make this delay as short as possible without making it so short that the volume function tracks low frequency sounds picked up by the microphone. Voice microphones have little signal sensitivity below about 150 Hz and the 8 millisecond delay has been found effective. Within the scope of this invention, the duration of signal that is averaged (time period) might fall anywhere between about 4 milliseconds, if there are no low frequencies that would be tracked by such short averaging, and about 100 milliseconds, which is about the outer limit of tolerable delays. A range between 6 and 20 milliseconds is preferred.

This smoothed output Es (representing volume over a short time period) is then passed through a non-linear lookup table 48 which, in one embodiment, is a combination of an amplitude compression function for medium to high level signals and an expansion function for low level signals which suppresses the low level signals relative to the medium and high level signals. This non-linear lookup table is a general purpose 16 bit in/out lookup table for which any mapping function can be inserted and used for optimizing the contrast between low level signals and medium or high level signals. The medium and high level signals are compressed to improve intelligibility and avoid overloading the circuit components or the hearing of the listeners.

Each output from the look up table specifies an amplification level to be applied to the signal. Because the outputs are binary values, there is a discontinuity from one value to the next. However, the jump from one value to the next in the look up table is chosen so that, when many consecutive values are taken together, the points of the values define a curving line that has no discontinuities (no sudden jumps) and no sudden bends (curves smoothly). The lack of sudden jumps and sudden bends yields better sound to the listener.

A variable manual control 43 gives the user a choice of how much of the original input signal X they wish to hear blended with the noise suppression control of the input signal X. A comparator circuit 50 uses the maximum of either the energy signal E or the static input level control set by the user which is then directly multiplied times the input signal to obtain the output noise suppressed signal Y. Because the maximum of these two sources is used, when the user sets the input high, there is no input signal modification; when the user sets the input low, there is full input signal modification; and when the user sets the input medium, the low level signal expansion (suppression) is less effective compared to when the user sets the input low. However, the effect of setting to medium causes the lower level signals to still get multiplied by a smaller number than full scale volume while still allowing the medium to high voice signals to pass at their envelope tracked higher volumes. The highest volumes are still compressed and the mid level volumes are boosted which gives rise to better intelligibility.

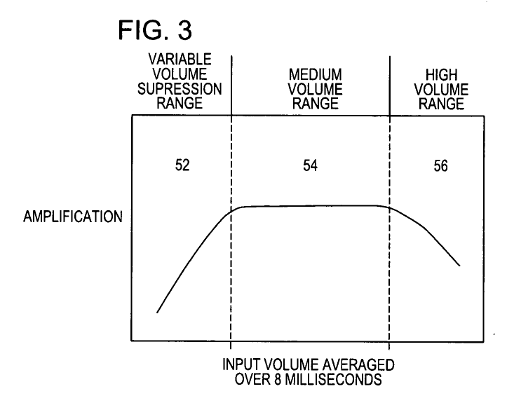

FIG. 3 is a graph showing level of amplification as a function of input signal volume averaged over the prior 8 milliseconds. Signals within a low range 52 receive less amplification than signal within a higher range 54. The width of the low range that receives reduced amplification can be adjusted by the variable manual control 43. Signals within a high range 56 also receive less amplification than signals within a medium volume range 54. The smooth curve shown in FIG. 3 is implemented with the output values of the look up table 48. Note that the curve shows neither sudden jumps nor sudden bends.

The circuit of this invention can be used wherever ambient noise is a problem, including motorcycles, factories, stock trading floors, etc. The scope of the invention should not be taken as specified or limited by the discussion above but rather as specified by the following claims.

We claim:

1 A fast-acting noise suppression device with a variable manual control of noise suppression, comprising:
(a) an input circuit operable to receive an electronic signal representing sound including noise;
(b) an output circuit operable to output an electronic signal representing sound;
(c) a noise suppression circuit coupled between the input circuit and the output circuit operable to effect variable amplification levels on the input signal as determined over time periods between 4 milliseconds and 100 milliseconds such that periods with volume levels within a low volume suppression range receive a relatively lower amplification than the periods with higher volume levels; and
(d) a variable manual control operable to adjust the low volume suppression range.

2 The noise suppression device of claim 1 where the amplification level varies continuously as a function of input volume level.

3. The noise suppression device of claim 2 where the amplification level varies smoothly as a function of input volume level

4 The noise suppression device of claim 1 further comprising a high volume level compression circuit operable to effect variable amplification levels on the input signal as determined over time periods between 4 milliseconds and 100 milliseconds such that periods with volume levels higher than a medium volume level range receive a lower amplification than periods with a medium volume level range.

5 The noise suppression device of claim 4 where the amplification level varies continuously as a function of input volume level.

6. The noise suppression device of claim 5 where the amplification level varies smoothly as a function of input volume level

7. The noise suppression device of claim 1 further comprising an input level adjusting circuit operable to adjust sensitivity of the input volume determination by adjusting the level of the input signal before the volume level is determined.

8 The noise suppression device of claim 1 where the time period is between 6 and 20 milliseconds.

9. A fast-acting noise suppression device with compression of high volume levels, comprising:
(a) an input circuit operable to receive an electronic signal representing sound including noise;
(b) an output circuit operable to output an electronic signal representing sound;
(c) a noise suppression circuit coupled between the input circuit and the output circuit operable to effect variable amplification levels on the input signal as determined over time periods between 4 milliseconds and 100 milliseconds such that periods with volume levels within a low volume suppression range receive a relatively lower amplification than the periods with higher volume levels; and
(d) a high volume level compression circuit operable to effect variable amplification levels on the input signal as determined over time periods between 4 milliseconds and 100 milliseconds such that periods with volume levels higher than a medium volume level range receive a lower amplification than periods with a medium volume level range.

10. The noise suppression device of claim 9 where the amplification level varies continuously as a function of input volume level.

11. The noise suppression device of claim 10 where the amplification level varies smoothly as a function of input volume level.

12. The noise suppression device of claim 9 further comprising an input level adjusting circuit operable to adjust sensitivity of the input volume determination by adjusting the level of the input signal before the volume level is determined.

13. The noise suppression device of claim 9 where the time period is between 6 and 20 milliseconds.

14. A fast-acting noise suppression device with a variable noise suppression using a look up table, comprising:
(a) an input circuit operable to receive an electronic signal representing sound including noise;
(b) an output circuit operable to output an electronic signal representing sound;
(c) a noise suppression circuit coupled between the input circuit and the output circuit operable to effect variable amplification levels on the input signal as determined over time periods between 4 milliseconds and 100 milliseconds such that periods with volume levels within a low volume suppression range receive a relatively lower amplification than the periods with higher volume levels; and
(d) where the variable amplification level is determined by applying the input volume level over a time period to a look up table.

15. The noise suppression device of claim 14 where the amplification level as determined via the look up table varies continuously as a function of input volume level.

16. The noise suppression device of claim 15 where the amplification level as determined via the look up table varies smoothly as a function of input volume level

17. The noise suppression device of claim 14 wherein the look up table contains values for high input volume levels such that periods with volume levels higher than a medium volume level range receive a lower amplification than periods with a medium volume level range.

18. The noise suppression device of claim 14 further comprising an input level adjusting circuit operable to adjust sensitivity of the input volume determination by adjusting the level of the input signal before the volume level is determined.

19. The noise suppression device of claim 14 where the time period is between 6 and 20 milliseconds.
